(19) United States
(12) Patent Application Publication
(10) Pub. No.: US 2007/0123936 A1
(43) Pub. Date: May 31, 2007

(54) Arterial Closure Button

(75) Inventors: Mark Goldin, Orlando, FL (US); William Christy, Winter Park, FL (US); Donald Stoner, Ormond Beach, FL (US)

Correspondence Address: FROST BROWN TODD, LLC, 2200 PNC CENTER, 201 E. FIFTH STREET, CINCINNATI, OH, 45202, US

(73) Assignee: AOI Medical, Inc., Orlando, FL (US)

(21) Appl. No.: 11/559,972

(22) Filed: Nov. 15, 2006

Related U.S. Application Data
(60) Provisional application No. 60/736,813, filed on Nov. 15, 2005.

(51) Int. Cl. A61B 17/04

(52) U.S. Cl. 606/232

(57) ABSTRACT

Provided is a medical device for restoring the integrity of vessels comprising a closure button insertable therein, where the closure button is adapted to plug a hole or port in a vessel to prevent blood loss, for use in an intervertebral disc herniation, or for use in a crushed vertebral body.

### PRIORITY

The application claims priority from the disclosure of U.S. Provisional Patent Application Ser. No. 60/736,813, entitled “Arterial Closure Button,” filed Nov. 15, 2005, which is herein incorporated by reference in its entirety.

### BACKGROUND

Versions of the present invention relate to restoring the integrity of vessels and, more particularly, to restoring the integrity of arteries and veins with a closure button. A variety of medical devices have been created and used, but no one prior to the inventor(s) has created or used the invention described in the appended claims.

### BRIEF DESCRIPTION OF THE FIGURES

In accordance with versions herein, it is believed the present invention will be better understood from the following description taken in conjunction with the accompanying drawings. The drawings and detailed description that follow are intended to be merely illustrative and are not intended to limit the scope of the invention.

FIG. 1 presents a cross-sectional view of an insertion tube having a closure button inserted therein shown inserted into a vessel before deployment of the closure button;

FIG. 2 presents a cross-sectional view of the closure button of FIG. 1 shown deployed within a vessel;

FIG. 3 presents a cross-sectional view of the closure button of FIG. 1 shown being pulled against the inner wall of the vessel while the insertion tube of FIG. 1 is pushed against the outer wall of the vessel;

FIG. 4 presents a perspective view of one version of a closure button having traction spikes thereon;

FIG. 5 presents a cross-sectional view of the closure button of FIG. 4 taken along line 5-5 shown with a central projection having an eyelet therethrough adapted to receive a suture;

FIG. 6 presents a cross-sectional view of one version of a closure button having a retention member coupled thereto.

FIG. 7 presents a cross-sectional view of one version of a closure button being used in an intervertebral application.

FIG. 8 presents a cross-sectional view of one version of a closure button being used in a vertebra.

### DETAILED DESCRIPTION OF THE INVENTION

Versions of a closure button disclosed herein are adapted to seal or restore the integrity of veins and arteries. For example, the closure button may be used to seal a femoral artery that has been used for access during a cardiac catheter procedure. During such a procedure, access is generally gained by way of an 18 g needle or 4F to 6F stylet/dilators. After the procedure the femoral artery needs to be closed. In one version, the closure button is a permanently implanted device that seals the opening in the arterial wall and prevents blood loss therethrough.

The closure button may be configured in any suitable shape such as, for example, an oval shape, a circular shape, an umbrella-like shape, a multi-sided shape, a tube, a shape matching the internal structure of a bone or a vessel, or combinations thereof. The closure button may also be, for example, an inflatable balloon. The closure button may be configured from any suitable material such as, for example, a polymeric material. The closure button may have a memory retention capability where, upon deployment, the closure button will return to its native state. In a further version, the closure button may open with any suitable spring or hinge system that may be manually or automatically deployed upon insertion into a vessel.

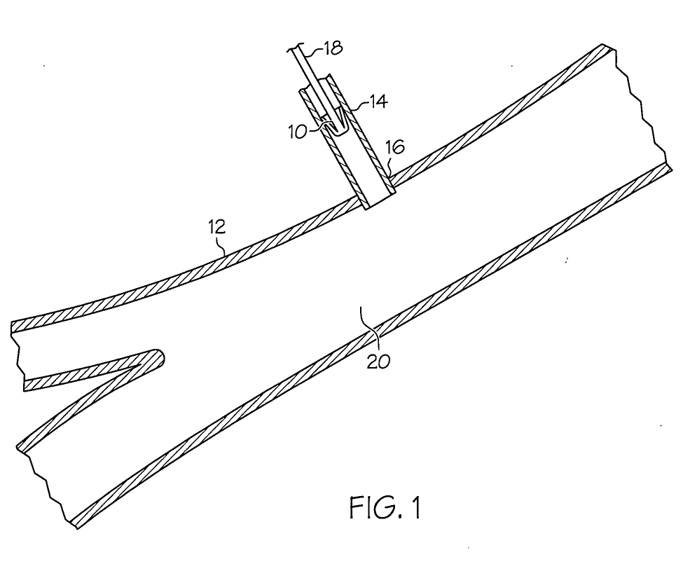

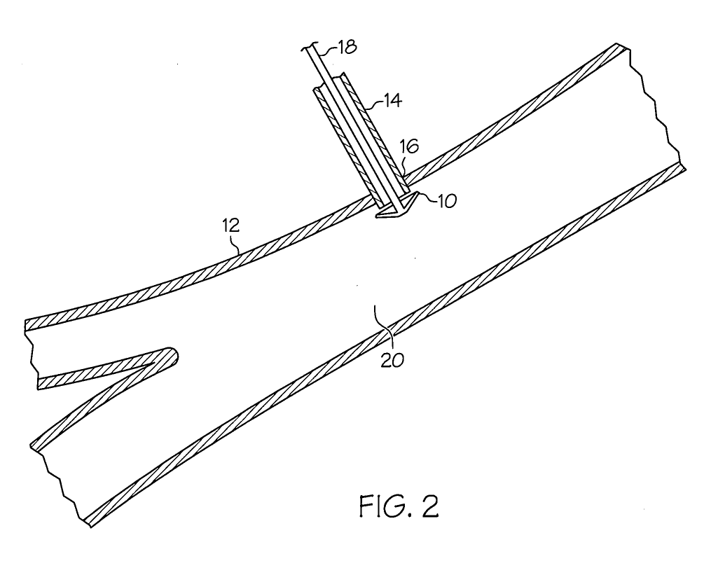

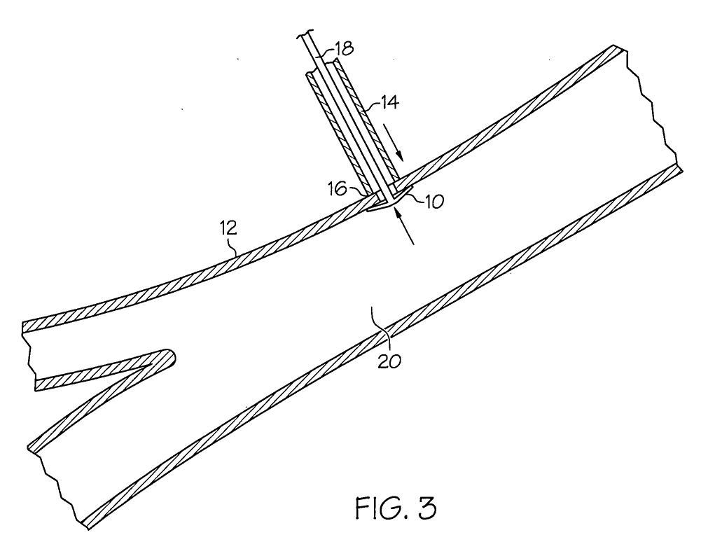

Referring to FIGS. 1-3, in one version the closure button 10 is configured from an elastomeric material and is folded into an insertion tube 14. The insertion tube 14 is then inserted into the hole or access port 16 in an artery, vein, or vessel 12. Upon insertion, the closure button 10 may be pushed distally, with a rod 18, or the like, out of the insertion tube 14 and into the lumen 20 of the vessel 12. Once pushed out of the insertion tube 14, the elastomeric closure button 10 may be deployed and may expand within the vessel 20. After deployment, the closure button 10 may be pulled adjacent the vessel 12 wall, thereby restoring the integrity of the vessel 12 and/or blocking the flow of blood therethrough. In one version, the natural internal pressure of the vessel 12 may used to hold the closure button 10 flat or substantially flat against the outer wall.

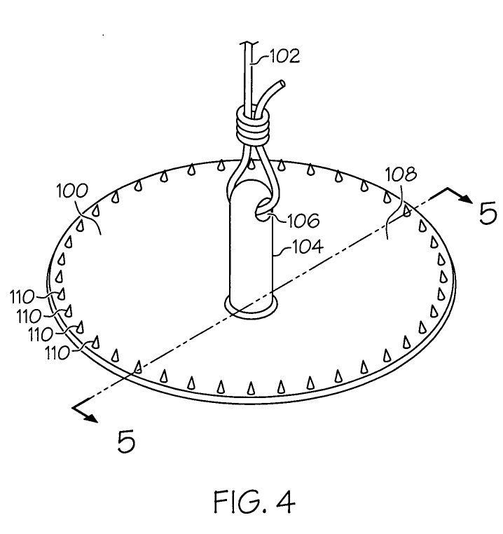

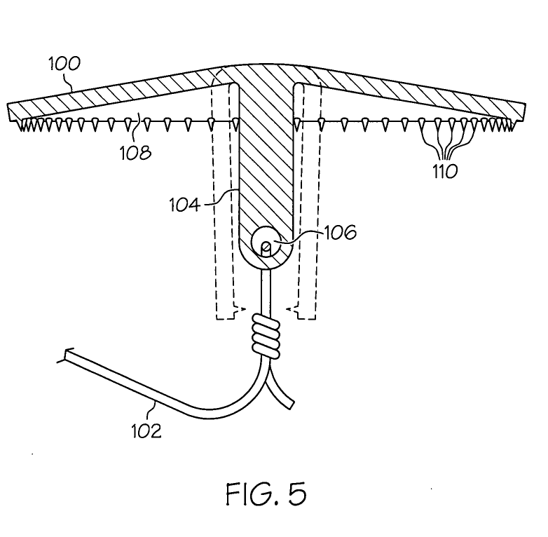

In an alternate version, referring to FIGS. 4-5, the closure button 100 may be tethered or otherwise secured to the arterial wall with, for example, a suture 102. In the illustrated version, the closure button 100 includes a central projection 104 having an eyelet 106 therethrough where, for example, a tether or suture 102 may be threaded through the eyelet 106 and sutured to the skin of the patient or to pull the closure button 100 against the vessel wall. It is contemplated that the tether and/or closure button may be constructed from a bioabsorbable material that will disintegrate within the body over time.

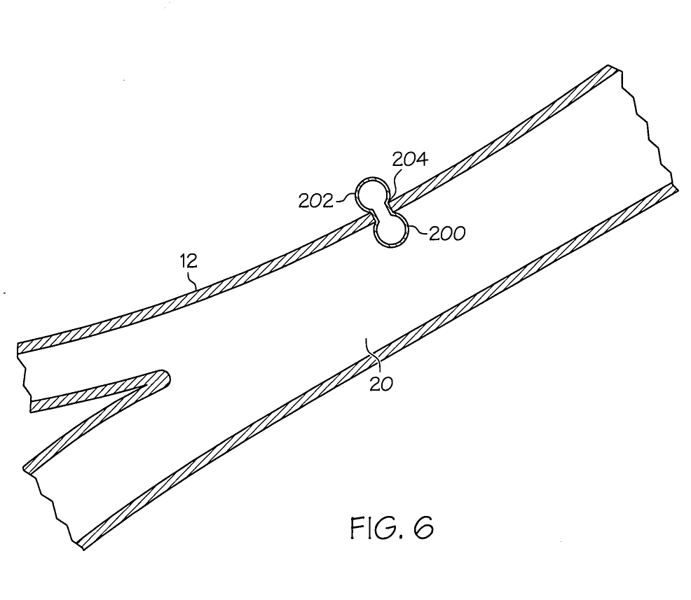

Referring to FIG. 6, the closure button 200 may include a retention member 202 coupled with the closure button 200, where the closure button 200, the retention member 202, and the coupling 204 therebetween resemble a spool or dumb bell shape. The retention member 202 includes any suitable mode, manner, or device for securing the closure button 200 in place and may be, for example, a round or oval disk positioned adjacent to the vessel 12 wall. The retention member 202 may apply pressure to the outer wall of the vessel 12 as needed to ensure the closure button 200 is against the interior wall and/or to prevent slippage.

Referring back to FIGS. 4-5, the closure button 100 may be, for example, folded and deployed in the shape of an umbrella. The surface 108 of the closure button 100 that is in contact with the interior vessel 12 wall may be rough, contain spikes, traction members 100, or the like, to prevent slippage off of the vessel 12 wall. It will be appreciated that any suitable surface or projection increasing the friction and/or connection between closure button 100 and the vessel 12 wall is contemplated. It will be appreciated that any suitable combination of elements disclosed herein is contemplated.

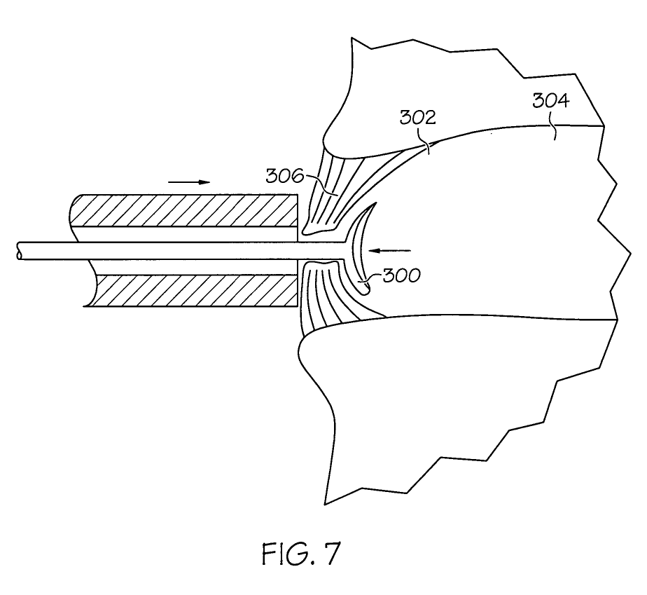

Referring to FIG. 7, it will be appreciated that a closure button 300 may be used in any suitable field including for herniated discs. As illustrated, the closure button 300 may be inserted into the nucleus 304 of a vertebral disk 302 through the fibrous annular ring 306. The closure button 300 may be inserted in accordance with versions described herein to reduce the effect of the herniation, to repair the herniation, or for any other suitable therapeutic benefit.

Referring to FIG. 8, it will be appreciated that the closure button may be used in one or a plurality of vertebra. As illustrated, the closure button may be used for a fractured, crushed, and/or collapsed vertebra. The closure button may be used as a temporary seal preceding the placement or delivery of a fluid, such as bone cement, into a vertebra. The placement of the closure button may prevent or diminish the amount of bone cement permitted to leak therefrom. After the material is set, the closure button may be left in place or may be removed.

What is claimed is:

1. a medical device comprising:
(a) a first member, the first member extending laterally about a central axis, wherein the first member is operably configured to engage tissue about an aperture such that the aperture is substantially closed;
(b) a second member, the second member extending axially along the central axis, wherein a first end of the second member is fixed to the first member; and
(c) a connection member, the connection member being associated with the second end of the second member, wherein the connection member is operably configured to urge the first member and the second member axially along the central axis such that the aperture is substantially closed.

2. The medical device of claim 1, wherein the tissue is a vessel having an aperture, the closure of which is desirable.

3. The medical device of claim 1, wherein the tissue is a herniated intervertebral disc.

4. The medical device of claim 1, wherein the first member is flexible.

5. The medical device of claim 4, wherein the first member is substantially disk-shaped.

6. The medical device of claim 4, wherein the first member comprises a plurality of tissue retention members.

7. The medical device of claim 6, wherein the plurality of tissue retention members are radially positioned projections.

8. The medical device of claim 4, wherein the first member and the second member are operably configured for placement within a delivery lumen.

9. a medical device comprising:
(a) a first member, the first member extending laterally about a central axis, wherein the first member is operably configured to engage tissue at about an aperture such that the aperture is substantially closed;
(b) a second member, the second member extending laterally about the central axis, wherein the first member is operably configured to engage the tissue such that the aperture is substantially closed; and
(c) a connection member, the connection member operably configured to join the first member with the second member in a spaced apart configuration, wherein the first member is configured for placement on a first side the tissue and the second member is configured for placement on a second side of the tissue such that the connection member passes therebetween.

10. The medical device of claim 9, wherein the tissue is a vessel having an aperture the closure of which is desirable.

11. The medical device of claim 9, wherein the tissue is a herniated intervertebral disc.

12. The medical device of claim 9, wherein the first member and the second member are flexible.

13. The medical device of claim 12, wherein the first member and the second member are operably configured for placement within a delivery lumen.

14. A method for closing a tissue aperture comprising:
providing a medical device comprising;
(a) a first member, the first member extending laterally about a central axis, wherein the first member is operably configured to engage tissue at about an aperture such that the aperture is substantially closed;
(b) a second member, the second member extending laterally about the central axis, wherein the first member is operably configured to engage tissue such that the aperture is substantially closed; and
(c) a connection member, the connection member operably configured to join the first member with the second member in a spaced apart configuration, wherein the first member is configured for placement on a first side the tissue and the second member is configured for placement on a second side of the tissue such that connection member passes therebetween;
providing an insertion tube;
deforming the medical device such that it is housed within the insertion tube;
inserting the insertion tube through the aperture of the tissue;
deploying the first member of the medical device on the first side of the tissue;
deploying the second member of the medical device on the second side of the tissue;
removing the insertion tube; and
substantially closing the aperture with the medical device.

15. The method of claim 14, wherein the tissue is a vessel having an aperture the closure of which is desirable.

16. The method of claim 14, wherein the tissue is a herniated intervertebral disc.
